(19) United States
(12) Patent Application Publication
(10) Pub. No.: US 2022/0061711 A1
(43) Pub. Date: Mar. 3, 2022

(54) TEST SUPPORT APPARATUS, TEST SUPPORT METHOD, AND TEST SUPPORT PROGRAM

(71) Applicant: FUJIFILM CORPORATION, Tokyo (JP)

(72) Inventors: Haruyasu NAKATSUGAWA, Kanagawa (JP); Tomohide HIRAGAMI, Kanagawa (JP); Yasuhisa KANEKO, Kanagawa (JP); Kenji NAGAMIYA, Kanagawa (JP); Yasuyuki HOSONO, Kanagawa (JP); Nobuya KITAMURA, Kanagawa (JP)

(21) Appl. No.: 17/407,207

(22) Filed: Aug. 20, 2021

(30) Foreign Application Priority Data
Aug. 27, 2020 (JP) 2020-143522

(51) Int. Cl. A61B 5/145; A61B 5/00

(52) U.S. Cl. CPC A61B 5/14532; A61B 5/681; A61B 5/7275; A61B 5/74

(57) ABSTRACT

A test support apparatus including at least one processor, wherein the processor is configured to predict a timing at which a postprandial hyperglycemic spike occurs by monitoring a variation in a blood glucose equivalent value of a user that correlates with a blood glucose value, and provide a notification of at least one blood sampling timing for measuring the blood glucose value based on a result of the prediction.

### CROSS-REFERENCE TO RELATED APPLICATIONS

The present application claims priority under 35 U.S.C. § 119 to Japanese Patent Application No. 2020-143522, filed on Aug. 27, 2020. The above application is hereby expressly incorporated by reference, in its entirety, into the present application.

### BACKGROUND

### Technical Field

The technique of the present disclosure relates to a test support apparatus, a test support method, and a test support program.

### Related Art

In recent years, a method of monitoring biological information such as a pulse, a blood pressure, respiration, an electrocardiogram, maximum oxygen intake, a blood glucose equivalent value, and a body temperature by using a wearable terminal such as a smart watch and managing health improvement and disease prevention based on the monitored biological information is beginning to spread. In particular, in order to monitor a glucose value that correlates with a blood glucose value of a diabetic patient, a measurement device which automatically and continuously measures a glucose value of the patient at predetermined time intervals by using a sensor or the like attached to a skin of the patient is known (refer to, for example, JP2017-515520A). In addition, self monitoring of blood glucose (SMBG) by blood sampling has been performed such that diabetic patients manage their blood glucose values. Further, as a measurement device for performing self monitoring of blood glucose, for example, as described in JP2016-133890A, a measurement device that measures a blood glucose value by providing blood obtained by puncturing a finger tip of a patient to a sensor is known.

On the other hand, although a patient can recognize a tendency of a change in the blood glucose value by continuously monitoring the blood glucose equivalent value such as the glucose value by using a measurement device, there may be a deviation between the blood glucose equivalent value and the blood glucose value obtained by blood sampling. That is, the blood glucose equivalent value may be higher or lower than the blood glucose value obtained by blood sampling. For this reason, assuming that a patient tries to determine the tendency of the blood glucose value only by the blood glucose equivalent value, in a case where the blood glucose equivalent value is high, the patient has extra anxiety, and in a case where the blood glucose equivalent value is low, further monitoring of the blood glucose equivalent value is required. In order to solve such a problem, a method of presenting a timing for performing measurement by blood sampling to a patient has been proposed based on a comparison result between information on a measurement result and a measurement time, which are continuously obtained by a measurement device, and a predetermined threshold value of the blood glucose value and a measurement period (refer to, for example, JP2019-018005A). In the method described in JP2019-018005A, a timing at which a postprandial hyperglycemic spike or hypoglycemia occurs is calculated in advance based on continuous measurement results obtained by a measurement device, and the calculated timing is presented to a patient, as a timing for performing measurement by blood sampling. According to the method described in JP2019-018005A, a patient performs measurement by blood sampling according to the presented timing for blood sampling.

### SUMMARY

In the method described in JP2019-018005A, the measurement timing for blood sampling is presented to a patient in advance by using the continuous measurement results obtained by the measurement device. As a result, the presented measurement timing may deviate from the actual timing after a meal. Here, the postprandial hyperglycemic spike is a symptom seen in an early stage of diabetes, and is a symptom in which the blood glucose value increases approximately one hour to two hours after a meal even in a case where a fasting blood glucose value is within a normal range. In a case where the postprandial hyperglycemic spike is neglected, blood vessels are damaged and complications of arteriosclerosis and diabetes are more likely to progress. It is also considered that complications such as myocardial infarction, angina, and stroke are more likely to progress. For this reason, it is necessary to manage the blood glucose value by performing blood sampling that is targeted at the timing at which a postprandial hyperglycemic spike occurs.

The present disclosure has been made in view of the above circumstances, and an object of the present disclosure is to perform blood sampling that is targeted at a timing at which a postprandial hyperglycemic spike occurs.

There is provided a test support apparatus according to an aspect of the present disclosure including at least one processor configured to predict a timing at which a postprandial hyperglycemic spike occurs by monitoring a variation in a blood glucose equivalent value of a user that correlates with a blood glucose value, and provide a notification of at least one blood sampling timing for measuring the blood glucose value based on a result of the prediction.

In the test support apparatus according to the aspect of the present disclosure, the processor may be configured to provide a notification of at least one blood sampling timing for measuring the blood glucose value by using, as a reference, the timing at which the postprandial hyperglycemic spike occurs.

In the test support apparatus according to the aspect of the present disclosure, the timing at which the postprandial hyperglycemic spike occurs may be a timing at which the postprandial hyperglycemic spike reaches a peak.

Further, in the test support apparatus according to the aspect of the present disclosure, the processor may be configured to predict the timing at which the postprandial hyperglycemic spike occurs by referring to a past monitoring result representing a variation in a past blood glucose equivalent value of the user.

Further, in the test support apparatus according to the aspect of the present disclosure, the processor may be configured to predict the timing at which the postprandial hyperglycemic spike occurs by referring to a past meal time recorded on a time axis of the past monitoring result.

Further, in the test support apparatus according to the aspect of the present disclosure, the processor may be configured to predict the timing at which the postprandial hyperglycemic spike occurs by comparing, in the past monitoring result, the variation in the blood glucose equivalent value from the past meal time with the variation in the blood glucose equivalent value from a latest meal time of the user.

Further, in the test support apparatus according to the aspect of the present disclosure, the processor may be configured to provide, in a case where it is predicted that the postprandial hyperglycemic spike does not occur within a preset time from the latest meal time, a notification that the postprandial hyperglycemic spike does not occur.

Further, in the test support apparatus according to the aspect of the present disclosure, the processor may be configured to predict the timing at which the postprandial hyperglycemic spike occurs based on a latest meal content of the user.

Further, in the test support apparatus according to the aspect of the present disclosure, the processor may be configured to predict the timing at which the postprandial hyperglycemic spike occurs by referring to the latest meal content of the user and a past meal content of the user.

Further, in the test support apparatus according to the aspect of the present disclosure, the processor may be configured to determine the latest meal content by using an image of a meal of the user.

Further, in the test support apparatus according to the aspect of the present disclosure, the processor may be configured to determine the past meal content by using an image of a meal of the user.

Further, in the test support apparatus according to the aspect of the present disclosure, the image of the meal is a moving image.

Further, in the test support apparatus according to the aspect of the present disclosure, the processor may be configured to provide a notification of plural the blood sampling timings for one timing at which the postprandial hyperglycemic spike occurs.

Further, in the test support apparatus according to the aspect of the present disclosure, the blood sampling timing may be a timing at which the user has to perform self blood sampling by himself/herself.

Further, in the test support apparatus according to the aspect of the present disclosure, the blood glucose equivalent value may be acquired by a measurement device attached to the user.

In this case, the measurement device may be a wearable device.

Further, the test support apparatus according to the aspect of the present disclosure may further include a measurement device that acquires the blood glucose equivalent value.

Further, in the test support apparatus according to the aspect of the present disclosure, the test support apparatus is a wearable apparatus.

There is provided a test support method according to another aspect of the present disclosure including: predicting a timing at which a postprandial hyperglycemic spike occurs by monitoring a variation in a blood glucose equivalent value of a user that correlates with a blood glucose value; and providing a notification of at least one blood sampling timing for measuring the blood glucose value based on a result of the prediction.

The test support method according to the aspect of the present disclosure may be provided as a program to be executed by a computer.

According to the present disclosure, it is possible to perform blood sampling that is targeted at the timing at which a postprandial hyperglycemic spike occurs.

### BRIEF DESCRIPTION OF THE DRAWINGS

FIG. 1 is a schematic diagram illustrating a configuration of a test support system to which a test support apparatus according to an embodiment of the present disclosure is applied.

FIG. 2 is a hardware configuration diagram of the test support apparatus according to the present embodiment.

FIG. 3 is a hardware configuration diagram of a wristwatch-type measurement device.

FIG. 4 is a hardware configuration diagram of the measurement device for measuring glucose in an interstitial fluid.

FIG. 5 is a functional configuration diagram of the test support apparatus according to the present embodiment.

FIG. 6 is a graph illustrating daily variations in blood glucose of a patient with type 2 diabetes.

FIG. 7 is a graph illustrating a past monitoring result of a user.

FIG. 8 is a graph for explaining prediction of a timing at which a postprandial hyperglycemic spike occurs.

FIG. 9 is a graph illustrating a past monitoring result of a user who does not eat breakfast on a daily basis.

FIG. 10 is a graph for explaining that peaks of postprandial hyperglycemic spikes deviate from each other between a blood glucose equivalent value and a blood glucose value.

FIG. 11 is a flowchart illustrating processing performed in the present embodiment.

FIG. 12 is a diagram illustrating a notification screen of a blood sampling timing.

FIG. 13 is a diagram for explaining measurement of a blood glucose value.

FIG. 14 is a diagram illustrating a notification screen of a blood sampling timing.

FIG. 15 is a diagram illustrating a notification screen for inquiring additional blood sampling.

FIG. 16 is a diagram illustrating a notification screen for urging a user to wear a measurement device for a longer time.

FIG. 17 is a diagram illustrating a notification screen in a case where a peak of a postprandial hyperglycemic spike is not predicted.

FIG. 18 is a graph illustrating a comparison result between a monitoring result of a blood glucose equivalent value and a test result obtained by blood sampling of a user.

### DESCRIPTION OF EMBODIMENTS

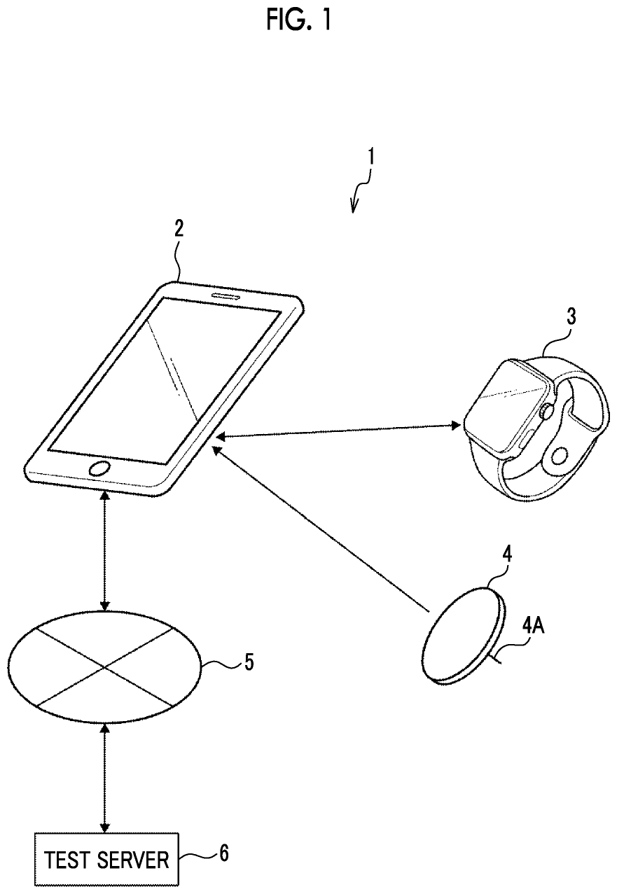

Hereinafter, embodiments of the present disclosure will be described with reference to the drawings. FIG. 1 is a schematic diagram illustrating a configuration of a test support system to which a test support apparatus according to an embodiment of the present disclosure is applied. As illustrated in FIG. 1, the test support system 1 according to the present embodiment includes a mobile terminal 2 such as a smartphone and measurement devices 3 and 4. The mobile terminal 2 and the measurement devices 3 and 4 can communicate with each other by short-range wireless communication such as Bluetooth (registered trademark). The mobile terminal 2 is connected to a test server 6 via a wired/wireless network 5 such that communication can be performed. The mobile terminal 2 is an example of the test support apparatus according to the present embodiment.

The measurement device 3 is a wristwatch-type wearable terminal such as a smart watch, and has a function of constantly measuring a blood glucose equivalent value of a user that correlates with a blood glucose value. The blood glucose equivalent value is biological information that correlates with the blood glucose value and is measured by a method that does not rely on blood sampling. The constant measurement means that the blood glucose equivalent value is automatically measured at a predetermined time interval, for example, 15 minutes, 30 minutes, or the like, without a measurement instruction from a user. The measurement device 3 may measure the blood glucose equivalent value even in a case where an instruction from a user is input while constantly measuring the blood glucose equivalent value. Further, the measurement device 3 may be a device that is worn by the user only at the time of measurement, such as a finger clip.

Further, the measurement device 3 is a non-invasive measurement device for the blood glucose equivalent value, and calculates the blood glucose equivalent value by, for example, irradiating the user with infrared rays and analyzing a signal emitted by glucose in blood. Alternatively, the measurement device 3 measures an electrocardiogram of the user, and calculates a blood glucose equivalent value that correlates with changes in the electrocardiogram. The measurement device 3 transmits the calculated blood glucose equivalent value to the mobile terminal 2.

The measurement device 4 is an invasive measurement device for the blood glucose equivalent value. For example, the measurement device 4 is attached to the user, constantly measures a glucose concentration in an interstitial fluid under epidermis of the user, and transmits the measured glucose concentration to the mobile terminal 2. For this reason, the measurement device 4 includes a needle-shaped filament 4A that is inserted under the epidermis of the user. The glucose concentration correlates with the blood glucose value, and thus the glucose concentration corresponds to the blood glucose equivalent value.

The user may possess any one of the measurement device 3 or the measurement device 4.

The mobile terminal 2 predicts a timing at which a postprandial hyperglycemic spike occurs by monitoring a variation in the blood glucose equivalent value measured by the measurement devices 3 and 4, and provides a notification of at least one blood sampling timing for measuring the blood glucose value based on a prediction result, specifically, using, as a reference, the timing at which a postprandial hyperglycemic spike occurs.

The postprandial hyperglycemic spike is a symptom seen in an early stage of diabetes, and is a symptom in which the blood glucose value significantly increases approximately one hour to two hours after a meal even in a case where a fasting blood glucose value is within a normal range. In a case where the postprandial hyperglycemic spike is neglected, blood vessels are damaged and complications of arteriosclerosis and diabetes are more likely to progress. It is also considered that complications such as myocardial infarction, angina, and stroke are more likely to progress. In the present embodiment, an occurrence of a postprandial hyperglycemic spike is predicted by monitoring a variation in the blood glucose equivalent value, and a blood sampling timing for measuring the blood glucose value based on a prediction result is notified.

The network 5 is a wide area network (WAN) that widely connects the mobile terminal 2 and the test server 6 via a public line network or a dedicated line network.

The test server 6 is provided in a test center that supports a test related to the blood glucose value. The test server 6 has a function of providing a test support program according to the present embodiment to the mobile terminal 2 or a function of providing information which is required when the mobile terminal 2 executes the test support program to the mobile terminal 2. In the test server 6, a software program for providing server functions to a general-purpose computer is installed.

The test center provides, to the user, various support for the test related to the blood glucose value. For example, the test center provides a test support program according to the present embodiment. Thus, the user is supported to perform the test. In addition, the test center also supports purchase of devices and test kits required for the test, and supports hospital test appointments. Further, the test center also performs a test using a specimen, which is obtained by self blood sampling of a user and is delivered by the user.

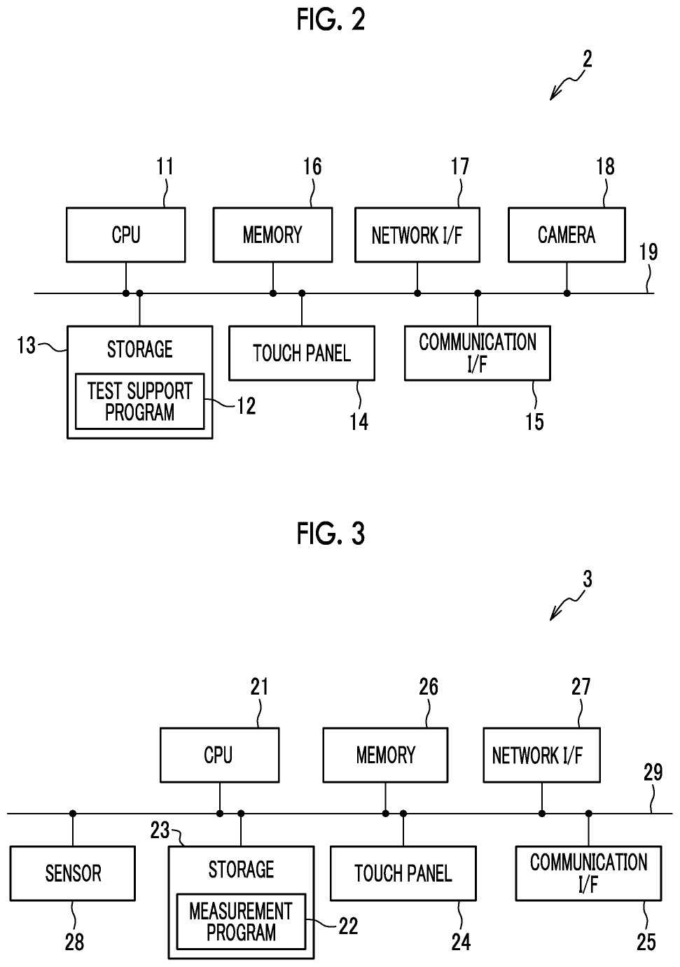

Next, the test support apparatus according to the present embodiment will be described. A hardware configuration of the test support apparatus according to the present embodiment will be described with reference to FIG. 2. As illustrated in FIG. 2, the mobile terminal 2 as the test support apparatus according to the present embodiment is a portable computer such as a smartphone, and includes a central processing unit (CPU) 11, a non-volatile storage 13, and a memory 16 as a transitory storage area. Further, the mobile terminal 2 includes a touch panel 14, a communication interface (I/F) 15 for short-range wireless communication, and a network I/F 17 wirelessly connected to the network 5. Further, the mobile terminal 2 includes a camera 18. The CPU 11, the storage 13, the touch panel 14, the communication I/F 15, the memory 16, the network I/F 17, and the camera 18 are connected to a bus 19. The CPU 11 is an example of a processor according to the present disclosure.

The storage 13 is realized by a solid state drive (SSD), a flash memory, or the like. A test support program 12 installed in the mobile terminal 2 is stored in the storage 13 as a storage medium. The CPU 11 reads out the test support program 12 from the storage 13, develops the test support program 12 in the memory 16, and executes the developed test support program 12.

The touch panel 14 is configured with a liquid crystal display, an organic EL, or the like, and performs various displays related to processing performed by the mobile terminal 2. The touch panel 14 also has a function as an input device for inputting various information to the mobile terminal 2.

The camera 18 acquires an image of a meal of the user by capturing the meal of the user according to, for example, an instruction of the user. The acquired image of the meal is stored in the storage 13. Alternatively, as described later, the acquired image of the meal is transmitted to the test server 6 together with the monitoring result of the blood glucose equivalent value according to an instruction of the user or without waiting for an instruction of the user.

The test support program 12 is stored in the test server 6 in a state of being accessible from the outside, and is downloaded and installed in the mobile terminal 2 in response to a request.

Next, the measurement device will be described. FIG. 3 illustrates a hardware configuration of the measurement device 3. The measurement device 3 is a wristwatch-type computer, and as illustrated in FIG. 3, includes a CPU 21, a non-volatile storage 23, and a memory 26 as a transitory storage area. Further, the measurement device 3 includes a touch panel 24, a communication I/F 25 for short-range wireless communication, a network I/F 27 wirelessly connected to an external network (not illustrated), and a sensor 28. The CPU 21, the storage 23, the touch panel 24, the communication I/F 25, the memory 26, the network I/F 27, and the sensor 28 are connected to a bus 29.

The storage 23 is realized by an SSD, a flash memory, or the like. A measurement program 22, which is installed in the measurement device 3 and is used to measure the blood glucose equivalent value, is stored in the storage 23 as a storage medium. The CPU 21 reads out the measurement program 22 from the storage 23, develops the measurement program 22 in the memory 26, and executes the developed measurement program 22.

The touch panel 24 is configured with a liquid crystal display, an organic EL, or the like, and performs various displays related to processing performed by the measurement device 3. The touch panel 24 also has a function as an input device for inputting various information to the measurement device 3.

The sensor 28 includes, for example, an infrared light source and an infrared detector, and detects a signal emitted by glucose in blood by irradiating a user who wears the measurement device 3 with infrared rays. The signal detected by the sensor 28 is analyzed by the CPU 21 that executes the measurement program 22, and thus the blood glucose equivalent value is calculated. Further, the sensor 28 may measure an electrocardiogram. In this case, the electrocardiogram is analyzed by the measurement program 22, and thus the blood glucose equivalent value is calculated.

The measurement program 22 is stored in the test server 6 in a state of being accessible from the outside, and is downloaded and installed in the measurement device 3 in response to a request. Alternatively, the measurement program 22 is downloaded in the mobile terminal 2, and then is downloaded and installed in the measurement device 3 via short-range wireless communication with the mobile terminal 2.

In the measurement device 3, the sensor 28 detects a signal emitted by glucose in blood at a predetermined time interval. The CPU 21 calculates the blood glucose equivalent value by analyzing the signal by the measurement program 22. Further, the CPU 21 transmits the calculated blood glucose equivalent value from the communication I/F 25 to the mobile terminal 2.

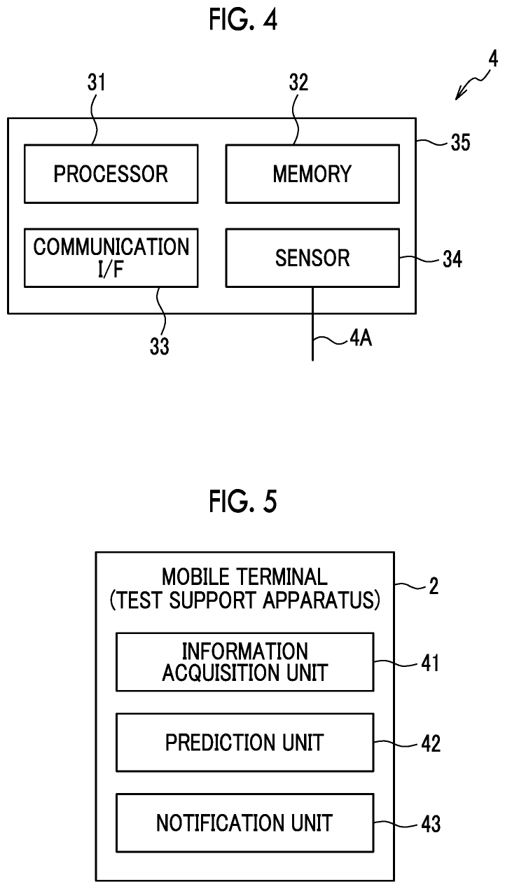

Next, the measurement device 4 will be described. FIG. 4 is a hardware configuration diagram of the measurement device 4. As illustrated in FIG. 4, the measurement device 4 is, for example, a measurement device described in JP2016-520379, which is attached to a human body and measures, as a blood glucose equivalent value, a glucose concentration in an interstitial fluid under epidermis. The measurement device 4 includes a processor 31, a memory 32 as a transitory storage area, a communication I/F 33 for short-range wireless communication, and a sensor 34. A needle-shaped filament 4A that is inserted under the epidermis is connected to the sensor 34. The processor 31, the memory 32, the communication I/F 33, and the sensor 34 are configured with an application specific integrated circuit (ASIC) 35 for measuring glucose in the interstitial fluid.

In the measurement device 4, the sensor 34 detects a signal indicating a glucose concentration in the interstitial fluid under the epidermis at a predetermined time interval or according to a measurement instruction by the user. The processor 31 calculates a glucose concentration by analyzing the signal. Further, the processor 31 transmits the calculated glucose concentration from the communication I/F 33 to the mobile terminal 2.

Next, a functional configuration of the test support apparatus according to the present embodiment will be described. FIG. 5 is a diagram illustrating a functional configuration of the test support apparatus according to the present embodiment, that is, the mobile terminal 2. As illustrated in FIG. 5, the mobile terminal 2 includes an information acquisition unit 41, a prediction unit 42, and a notification unit 43. The CPU 11 functions as the information acquisition unit 41, the prediction unit 42, and the notification unit 43 by executing the test support program 12. In the following description, it is assumed that the user possesses the measurement device 3 and monitors a variation in the blood glucose equivalent value measured by the measurement device 3 by using the mobile terminal 2.

The information acquisition unit 41 acquires the blood glucose equivalent value transmitted from the communication I/F 25 of the measurement device 3 by receiving the blood glucose equivalent value by the communication I/F 15. Since the blood glucose equivalent value is basically transmitted from the measurement device 3 at all times, that is, at predetermined time intervals, the information acquisition unit 41 constantly acquires the blood glucose equivalent value. The blood glucose equivalent value acquired by the information acquisition unit 41 is stored in the storage 13 in association with the acquisition date and time.

Further, in a case where the user eats a meal, the measurement device 3 may transmit a meal time to the mobile terminal 2. Instead of transmitting the meal time, the measurement device 3 may cause the mobile terminal 2 to input the meal time. Alternatively, an image of the meal may be captured by the camera 18 of the mobile terminal 2. In this case, the capturing time may be set as a meal time and may be used to assist a determination.

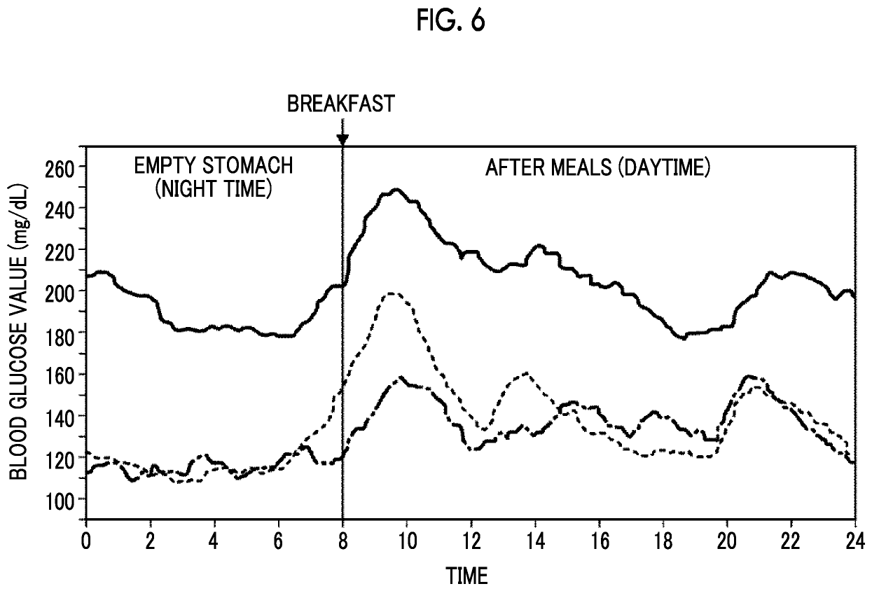

The prediction unit 42 predicts an occurrence of a postprandial hyperglycemic spike by monitoring a variation in the blood glucose equivalent value acquired by the information acquisition unit 41. Here, postprandial hyperglycemia will be described. FIG. 6 is a graph illustrating daily variations in blood glucose of a patient with type 2 diabetes. In FIG. 6, a horizontal axis represents a time of one day (24 hours), and a vertical axis represents a blood glucose value (mg/dL). A solid line represents a blood glucose value of a patient whose hemoglobin A1c (HbA1c) is equal to or higher than 9%. A broken line represents a blood glucose value of a patient whose HbA1c is equal to or higher than 7% and lower than 8%. A one-dot line represents a blood glucose value of a patient whose HbA1c is equal to or higher than 6.5% and lower than 7%. Further, FIG. 6 illustrates a variation in the blood glucose value in a case where the patient eats breakfast at around 8:00, eats lunch at around 12:00, and eats dinner at around 19:00. HbA1c is glycated hemoglobin in which hemoglobin as an erythrocyte component in blood is bonded with glucose. HbA1c represents a variation in the blood glucose for 1 to 2 months.

As illustrated in FIG. 6, the blood glucose value increases after meal regardless of a value of HbA1c. On the other hand, as HbA1c is higher, the postprandial hyperglycemic spike indicating that the blood glucose value greatly increases is particularly remarkable after breakfast. In order to make the postprandial hyperglycemic spike after breakfast easy to understand, in FIG. 6, a line is placed at a position indicating 8:00.

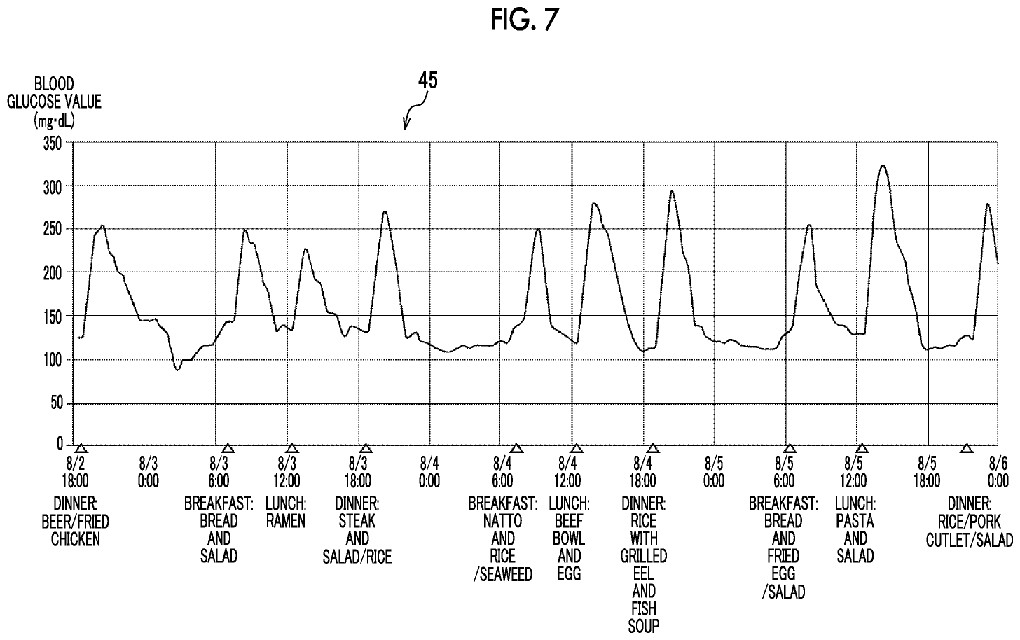

The prediction unit 42 predicts a timing at which the postprandial hyperglycemic spike occurs by monitoring the blood glucose equivalent value transmitted from the measurement device 3 and by referring to a past monitoring result representing a variation in the past blood glucose equivalent value of the user. FIG. 7 is a graph illustrating a past monitoring result of the user. In FIG. 7, a horizontal axis represents a time, and a vertical axis represents a blood glucose value (mg/dL). As illustrated in FIG. 7, the past monitoring result 45 represents a variation in the blood glucose equivalent value of the user that is measured by the measurement device 3 from 18:00 on Aug. 2, 2020 to 0:00 on Aug. 6, 2020. In addition, on the time axis, a triangular mark is recorded at a time when the user eats breakfast, lunch, and dinner. Further, meal content is registered for each meal. For example, bread and salad are registered for breakfast on August 3. In a case where the user acquires an image of the meal during the past meal, the meal content may be registered in the past monitoring result 45 by determining the meal content from the image of the meal. Further, the image of the meal may be registered in the past monitoring result 45.

In the past monitoring result 45 illustrated in FIG. 7, it can be seen that the postprandial hyperglycemic spike indicating that the blood glucose value increases occurs after three meals. Instead of the measurement by the measurement device 3, a blood glucose value of the user that is measured by blood sampling may be used as the past monitoring result.

The prediction unit 42 monitors the blood glucose equivalent value transmitted from the measurement device 3, and detects a timing at which the blood glucose equivalent value increases. Here, the blood glucose value increases with meal. On the other hand, the blood glucose value is maintained in a substantially constant value before meal. For this reason, the prediction unit 42 monitors whether or not a difference between the previous blood glucose equivalent value and the current blood glucose equivalent value is equal to or higher than a predetermined threshold value Th1. In a case where the difference between the previous blood glucose equivalent value and the current blood glucose equivalent value is equal to or higher than the predetermined threshold value Th1, it is determined that the blood glucose equivalent value measured this time tends to increase. On the other hand, the prediction unit 42 sets a time at which the previous blood glucose equivalent value is measured to a latest meal time closest to the current time. In a case where the meal time is transmitted from the measurement device 3 to the mobile terminal 2, the meal time may be set as the latest meal time.

In a case where it is determined that the blood glucose equivalent value tends to increase, the prediction unit 42 predicts a timing at which the postprandial hyperglycemic spike occurs by referring to the past monitoring result 45 of the user. At this time, the prediction unit 42 compares the meal time recorded on the time axis of the past monitoring result 45 with the latest meal time.

The prediction unit 42 specifies the past meal time closest to the latest meal time by referring to the past monitoring result 45. For example, in a case where the latest meal time is 8:00 am, the meal time is estimated to be breakfast. Thus, the prediction unit 42 specifies the past breakfast time in the past monitoring result 45. The prediction unit 42 calculates a time from the latest meal time to a time at which the postprandial hyperglycemic spike reaches a peak by referring to the variation in the blood glucose value at the specified past meal time.

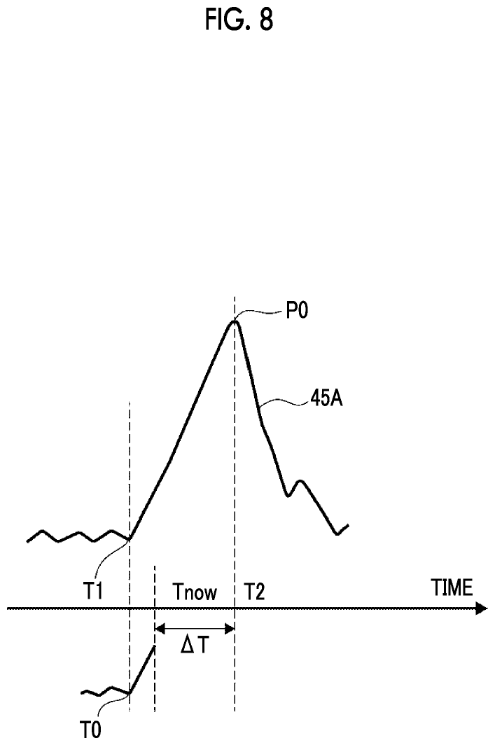

The past monitoring result 45 includes the variations in blood glucose value for three days. Thus, in a case of breakfast, the variations in the blood glucose value for three times are included. Therefore, the prediction unit 42 calculates a time from the three breakfasts included in the past monitoring result 45 to a time at which the postprandial hyperglycemic spike reaches a peak, and calculates, as a time from the breakfast to a time at which the postprandial hyperglycemic spike reaches a peak, an average value of the calculated times. FIG. 8 is a graph for explaining prediction of the timing at which the postprandial hyperglycemic spike occurs. FIG. 8 illustrates a profile 45A of an average value of the blood glucose values after three breakfasts in the past monitoring result 45. As illustrated in FIG. 8, the prediction unit 42 makes the meal time T1 in the profile 45A and the latest meal time T0 match with each other on a time axis. The prediction unit 42 calculates a time ΔT0 from the current time Tnow to a time T2 at which the postprandial hyperglycemic spike reaches a peak PO in the profile 45A. The prediction unit 42 calculates a timing at which ΔT is elapsed from the current time Tnow. The calculated timing is a timing at which the peak of the predicted postprandial hyperglycemic spike occurs.

In a case where the user captures an image of the meal using the camera 18 of the mobile terminal 2, the prediction unit 42 may determine the latest meal content (that is, the content of the image of the meal captured at a time point closest to the present time) based on the image of the meal, and predict a timing at which the peak of the postprandial hyperglycemic spike occurs using a determination result. Specifically, the prediction unit 42 compares the latest meal content with the meal content registered in the past monitoring result 45. In a case where the latest meal content and the registered meal content are similar, the prediction unit 42 uses the predicted timing as it is. On the other hand, as a result of comparing the latest meal with the registered meal content, in a case where the latest meal is low in carbohydrates, the blood glucose value slowly increases. Thus, the prediction unit 42 predicts a timing at which the peak of the postprandial hyperglycemic spike occurs by delaying the calculated timing by a predetermined time. For example, the prediction unit 42 predicts a timing at which the peak of the postprandial hyperglycemic spike occurs by adding a predetermined time ΔT3 to ΔT0.

Further, as a result of comparing the latest meal with the registered meal content, in a case where the latest meal is high in carbohydrates, the blood glucose value sharply increases. Thus, the prediction unit 42 predicts a timing at which the peak of the postprandial hyperglycemic spike occurs by preceding the calculated timing by a predetermined time. For example, the prediction unit 42 predicts a timing at which the peak of the postprandial hyperglycemic spike occurs by subtracting a predetermined time ΔT4 from ΔT0.

The prediction unit 42 may predict the timing at which the peak of the postprandial hyperglycemic spike occurs using only the latest meal content. Specifically, in a case where the latest meal content includes low carbohydrates, the blood glucose value slowly increases. Thus, the prediction unit 42 may predict a timing at which the peak of the postprandial hyperglycemic spike occurs by delaying the calculated timing by a predetermined time. On the other hand, in a case where the latest meal is high in carbohydrates, the blood glucose value sharply increases. Thus, the prediction unit 42 may predict a timing at which the peak of the postprandial hyperglycemic spike occurs by preceding the calculated timing by a predetermined time.

The image of the meal may be a still image or a moving image. In a case where the image of the meal is a moving image, the prediction unit 42 can recognize an eating order of the meals of the user. For example, the prediction unit 42 can recognize an order of the meals (whether vegetables are eaten first or a staple food is eaten first). Further, the prediction unit 42 can recognize a speed of eating (whether or not the user eats the meal fast). Therefore, the order of the meals or the speed of the meals can be reflected in the prediction of the timing at which the peak of the postprandial hyperglycemic spike occurs. For example, in a case where vegetables are eaten first, the blood glucose value slowly increases. Thus, the prediction unit 42 predicts a timing at which the peak of the postprandial hyperglycemic spike occurs by delaying the calculated timing by a predetermined time. On the other hand, in a case where a staple food is eaten first or eaten fast, the blood glucose value sharply increases. Thus, the prediction unit 42 predicts a timing at which the peak of the postprandial hyperglycemic spike occurs by preceding the calculated timing by a predetermined time.

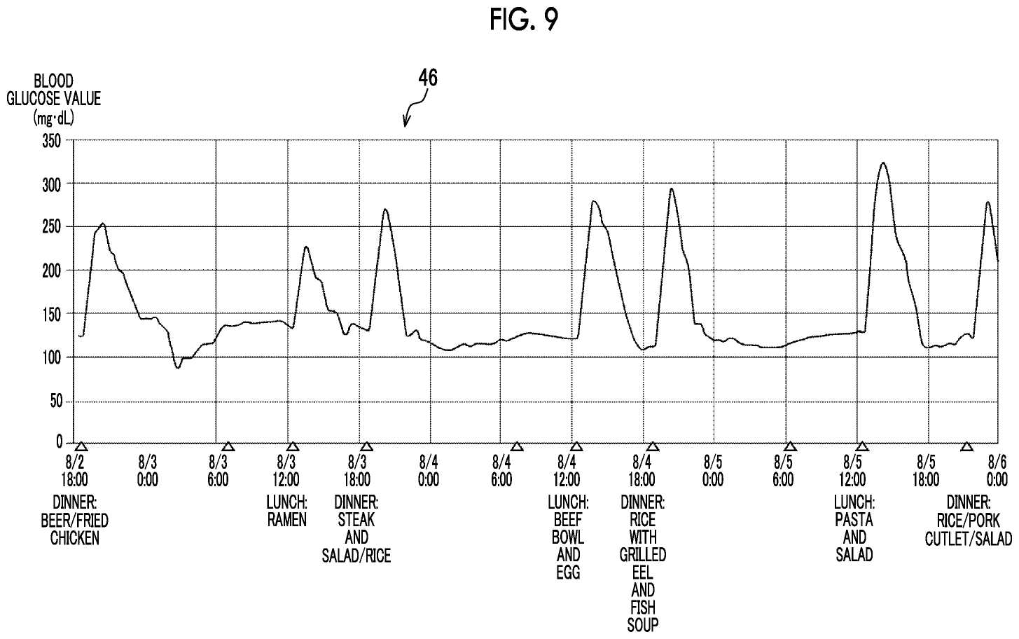

Here, FIG. 9 illustrates the past monitoring result of the blood glucose value of the user in a case where the user does not eat breakfast on a daily basis. As illustrated in FIG. 9, in a case where the user does not eat breakfast on a daily basis, in the past monitoring result 46, the postprandial hyperglycemic spike occurs only after lunch and after dinner, and the blood glucose value does not vary much during an estimated time after breakfast. In a case where the past monitoring result 46 of the user is as illustrated in FIG. 9, it is assumed that the prediction unit 42 detects the timing at which the blood glucose equivalent value increases. Further, it is assumed that the latest meal time which is set by the prediction unit 42 based on the variation in the blood glucose equivalent value is 8:00 am. The prediction unit 42 compares a variation in the blood glucose value around 8:00 am in the past monitoring result 46 with the blood glucose equivalent value during monitoring. On the other hand, the meal time is not registered in the past monitoring result 46. Therefore, the prediction unit 42 determines that the increase tendency of the blood glucose equivalent value at this time is not due to the postprandial hyperglycemic spike, and does not perform any notification.

Regardless of the presence or absence of registration of the meal time to the past monitoring results 45 and 46, at a time when an increase in the blood glucose equivalent value measured by the measurement device 3 is detected, the prediction unit 42 may determine whether or not a postprandial hyperglycemic spike occurs in the past monitoring results 45 and 46, and in a case where a postprandial hyperglycemic spike does not occur, may determine that an increase in the blood glucose equivalent value at this time is not due to the postprandial hyperglycemic spike. In this case, in a case where a postprandial hyperglycemic spike occurs in the past monitoring results 45 and 46, the prediction unit 42 may predict a timing at which a peak of the postprandial hyperglycemic spike occurs in the same manner as described above.

Further, even in a case where the user eats a snack, the blood glucose value increases. On the other hand, in this case, a postprandial hyperglycemic spike is less likely to occur as compared with a case where the user eats a meal. Therefore, in a case where the prediction unit 42 detects an increase in the blood glucose equivalent value due to eating of a snack by the user, the prediction unit 42 refers to a time when the blood glucose equivalent value increases in the past monitoring results 45 and 46. In a case where a postprandial hyperglycemic spike does not occur in the past monitoring results 45 and 46, the prediction unit 42 may determine that an increase tendency of the blood glucose equivalent value at this time is not due to a postprandial hyperglycemic spike.

In the present embodiment, before predicting the peak of the postprandial hyperglycemic spike, the prediction unit 42 may perform processing of determining whether or not an increase tendency of the blood glucose equivalent value is due to a postprandial hyperglycemic spike by referring to the past monitoring result of the user, and predicting a peak of the postprandial hyperglycemic spike in a case where the determination result is YES.

The notification unit 43 provides a notification of at least one blood sampling timing for measuring a blood glucose value based on the prediction result. Specifically, the notification unit 43 provides a notification of at least one blood sampling timing for measuring a blood glucose value by using, as a reference, a timing at which a postprandial hyperglycemic spike occurs. In the present embodiment, the blood sampling timing is a timing at which the user has to perform self blood sampling by himself/herself. The number of times the notification unit 43 provides a notification of the blood sampling may be set in advance by the user. Further, the notification unit 43 may set the number of times of notification of the blood sampling timing to any number. In this case, preferably, an upper limit of the number of times of notification is set, for example, to four times. The notification of the blood sampling timing is displayed on the touch panel 14 of the mobile terminal 2. On the other hand, information indicating the blood sampling timing may be transmitted from the mobile terminal 2 to the measurement device 3, and the measurement device 3 may provide a notification of the blood sampling timing.

Here, in a case where the number of times of notification is one time, the notification unit 43 provides a notification of the blood sampling timing such that the blood sampling timing matches with a time of the peak of the postprandial hyperglycemic spike. In a case where the number of times of notification is multiple times, the notification unit 43 provides a notification of the blood sampling timing at predetermined time intervals around the time of the peak of the postprandial hyperglycemic spike.

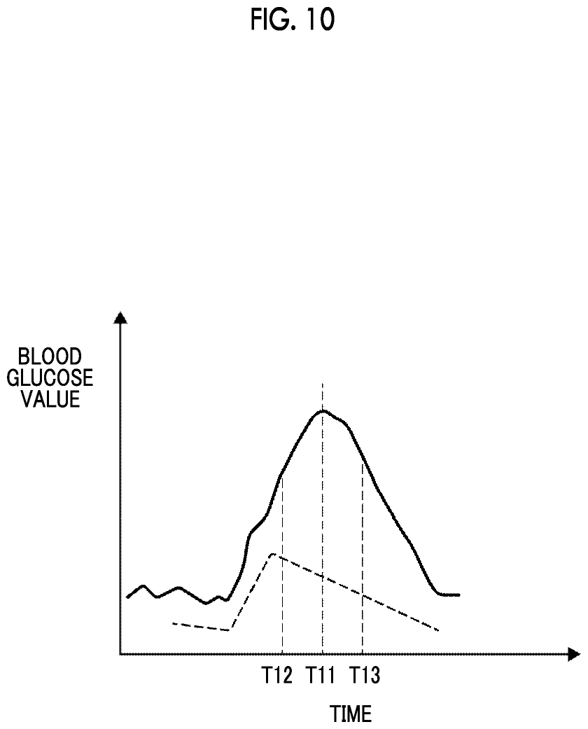

In a state where the postprandial hyperglycemic spike occurs, as illustrated in FIG. 10, a position of a peak of the blood glucose value which is obtained by blood sampling and is illustrated by a solid line and a position of a peak of the blood glucose equivalent value which is obtained by the measurement device 3 and is illustrated by a broken line may deviate from each other. For this reason, preferably, the prediction unit 42 predicts a timing of the peak of the postprandial hyperglycemic spike in consideration of the deviation. For example, in a case where the blood sampling timing is notified three times, preferably, the blood sampling timing is notified before, during, and after the peak of the postprandial hyperglycemic spike in consideration of the deviation. For example, as illustrated in FIG. 10, the blood sampling timing may be notified at a timing T11 at which the blood glucose value reaches the peak and at timings T12 and T13 before and after the timing T11 in consideration of the deviation.

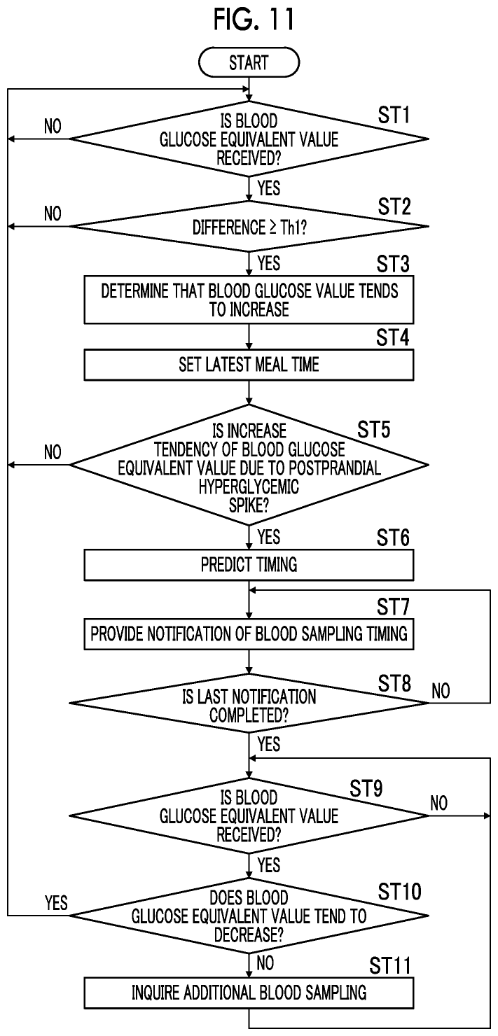

Hereinafter, processing performed in the present embodiment will be described. FIG. 11 is a flowchart illustrating processing performed in the present embodiment. In the present embodiment, it is assumed that the number of times the notification unit 43 provides a notification of the blood sampling timing is set to two times by the user. In order to monitor the blood glucose equivalent value, the information acquisition unit 41 monitors whether or not the communication I/F 15 receives the blood glucose equivalent value transmitted from the measurement device 3 (step ST1), and in a case where a monitoring result in step ST1 is YES, the prediction unit 42 determines whether or not a difference between the previous blood glucose equivalent value and the current blood glucose equivalent value is equal to or higher than a predetermined threshold value Th1 (step ST2). In a case where a determination result in step ST2 is NO, the process returns to step ST1. In a case where a determination result in step ST2 is YES, the prediction unit 42 determines that the blood glucose equivalent value measured at this time tends to increase (step ST3). In addition, the prediction unit 42 sets the latest meal time (step ST4).

Subsequently, the prediction unit 42 determines whether or not the increase tendency of the blood glucose equivalent value is due to the postprandial hyperglycemic spike by referring to the past monitoring result of the user (step ST5). In a case where a determination result in step ST5 is NO, the process returns to step ST1. In a case where a determination result in step ST5 is YES, the prediction unit 42 predicts a timing at which a peak of the postprandial hyperglycemic spike occurs (step ST6). Next, the notification unit 43 provides a notification of the blood sampling timing for measuring the blood glucose value based on a prediction result (step ST7).

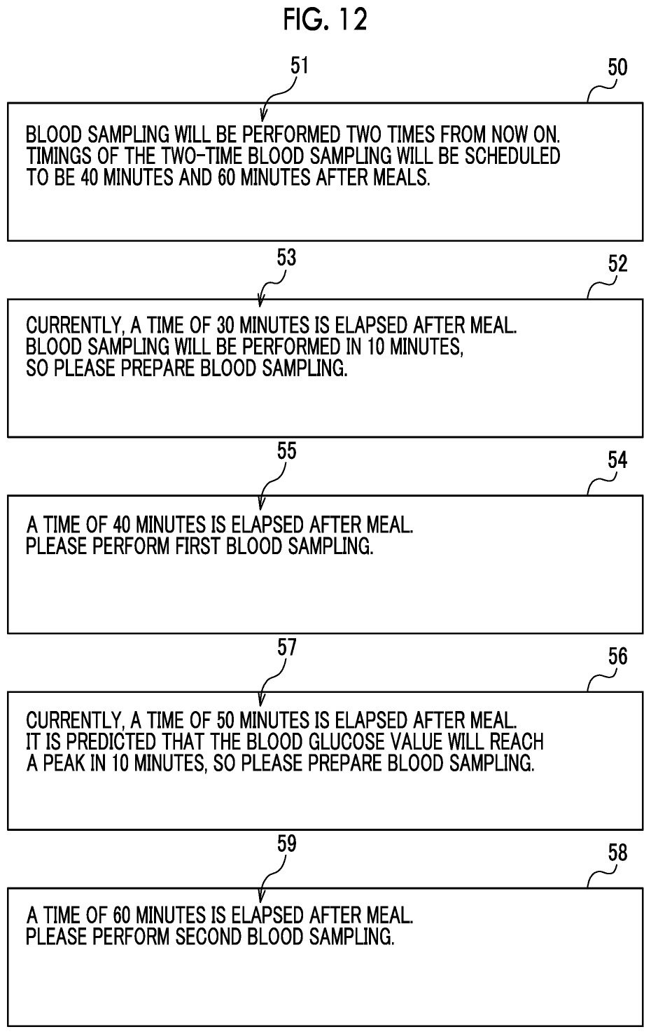

FIG. 12 is a diagram illustrating a notification screen of the blood sampling timing. In the present embodiment, the blood sampling timing is notified plural times. Thus, in FIG. 12, notification screens are illustrated in a time-series order from the top. On a notification screen 50 of the first blood sampling timing, the notification unit 43 displays a notification 51 indicating “Blood sampling will be performed two times from now on. Timings of the two-time blood sampling will be scheduled to be 40 minutes and 60 minutes after meal.”. From the notification screen 50, the user can recognize that the two-time blood sampling will be performed from now on.

After the notification screen 50 is displayed, the notification unit 43 counts a time from the latest meal time, and displays a notification screen 52 for providing a notification of the blood sampling timing in advance. As illustrated in FIG. 12, on the notification screen 52, a notification 53 indicating “Currently, a time of 30 minutes is elapsed after meal. Blood sampling will be performed in 10 minutes, so please prepare blood sampling.” is displayed. From the notification screen 52, the user can prepare blood sampling.

Further, the notification unit 43 counts a time from the latest meal time, and in a case where the time reaches a first blood sampling timing, displays a notification screen 54 for providing a notification that it is the blood sampling timing. As illustrated in FIG. 12, on the notification screen 54, a notification 55 indicating “A time of 40 minutes is elapsed after meal. Please perform first blood sampling.” is displayed.

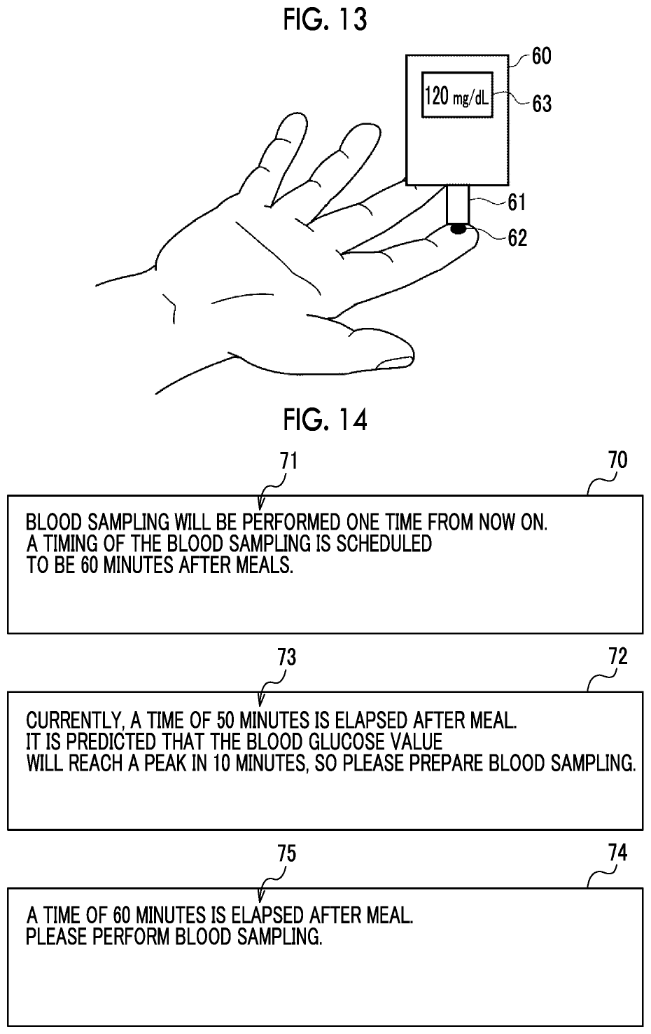

The user performs blood sampling at the timing when the notification screen 54 is displayed. The blood sampling by the user is self blood sampling. For the self blood sampling, first, the user makes his/her finger bleed by using a puncture device. As illustrated in FIG. 13, a tip of a sensor 61 of a measurement device 60 is brought into contact with blood 62, and thus the blood glucose value is measured. The measurement result is displayed on a display 63. The measured blood glucose value is transmitted from, for example, the measurement device 60 to the mobile terminal 2 or is input to the mobile terminal 2 by the user, and is stored in association with measurement time information.

After the first blood sampling, the notification unit 43 counts a time from the latest meal time, and displays a notification screen 56 for providing a notification of the second blood sampling timing in advance. As illustrated in FIG. 12, on the notification screen 56, a notification 57 indicating “Currently, a time of 50 minutes is elapsed after meal. It is predicted that the blood glucose value will reach a peak in 10 minutes, so please prepare blood sampling.” is displayed. From the notification screen 56, the user can prepare second blood sampling.

Further, the notification unit 43 counts a time from the latest meal time, and in a case where the time reaches a second blood sampling timing, displays a notification screen 58 for providing a notification that it is the blood sampling timing. As illustrated in FIG. 12, on the notification screen 58, a notification 59 indicating “A time of 60 minutes is elapsed after meal. Please perform second blood sampling.” is displayed. From the notification screen 58, the user can perform second blood sampling.

FIG. 14 illustrates a notification screen in a case where the number of times of the blood sampling which is set by the user is one time. As illustrated in FIG. 14, on a notification screen 70, a notification 71 indicating “Blood sampling will be performed one time. A timing of the blood sampling is scheduled to be 60 minutes after meal.” is displayed. From the notification screen 70, the user can recognize that one-time blood sampling will be performed from now on.

After the notification screen 70 is displayed, the notification unit 43 counts a time from the latest meal time, and displays a notification screen 72 for providing a notification of the blood sampling timing in advance. On the notification screen 72, a notification 73 indicating “Currently, a time of 50 minutes is elapsed after meal. It is predicted that the blood glucose value will reach a peak in 10 minutes, so please prepare blood sampling.” is displayed. From the notification screen 72, the user can prepare blood sampling.

Further, the notification unit 43 counts a time from the latest meal time, and in a case where the time reaches a blood sampling timing, displays a notification screen 74 for providing a notification that it is the blood sampling timing. On the notification screen 74, a notification 75 indicating “A time of 60 minutes is elapsed after meal. Please perform blood sampling.” is displayed. The user may perform blood sampling at the timing when the notification screen 74 is displayed.

After step ST7, the notification unit 43 determines whether or not a final notification is completed (step ST8). In a case where a determination result in step ST8 is NO, the process returns to step ST7.

Here, in a case where the blood glucose equivalent value continues to increase even after the blood sampling is completed, the postprandial hyperglycemic spike may not reach a peak. For this reason, in a case where a determination result in step ST8 is YES, the information acquisition unit 41 starts to monitor whether or not the communication I/F 15 receives the blood glucose equivalent value transmitted from the measurement device 3 (step ST9). In a case where a monitoring result in step ST9 is YES, the notification unit 43 determines whether or not the blood glucose equivalent value tends to decrease (step ST10). Whether or not the blood glucose equivalent value tends to decrease may be determined by determining whether or not the current blood glucose equivalent value is lower than the previous blood glucose equivalent value. In a case where a determination result in step ST10 is YES, the process returns to step ST1. In a case where a determination result in step ST10 is NO, the notification unit 43 outputs a notification inquiring whether or not to perform additional blood sampling (step ST11), and the process returns to step ST9.

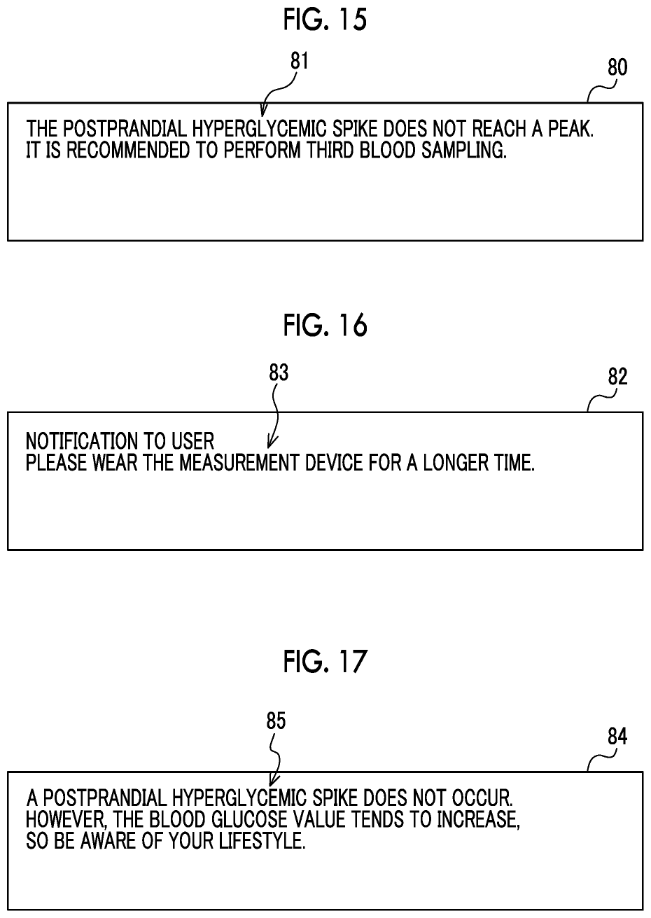

FIG. 15 is a diagram illustrating a notification screen for inquiring additional blood sampling. As illustrated in FIG. 15, on a notification screen 80, a notification 81 indicating “The postprandial hyperglycemic spike does not reach a peak. It is recommended to perform third blood sampling.” is displayed. From the notification screen 80, the user can perform third blood sampling by his/her own determination.

As described above, in the present embodiment, the mobile terminal 2 predicts a timing at which a postprandial hyperglycemic spike occurs by monitoring a variation in the blood glucose equivalent value which correlates with the blood glucose value, and provides a notification of at least one blood sampling timing for measuring the blood glucose value based on a prediction result, specifically, using, as a reference, the timing at which a postprandial hyperglycemic spike occurs. Therefore, it is possible to perform blood sampling that is targeted at the timing at which a postprandial hyperglycemic spike occurs.

Further, in a case where the timing at which the postprandial hyperglycemic spike occurs is the timing at which the postprandial hyperglycemic spike reaches a peak, it is possible to provide a notification of the blood sampling timing that is targeted at the peak of the postprandial hyperglycemic spike. Therefore, by performing blood sampling according to the blood sampling timing, the user can recognize the blood glucose value at the peak of the postprandial hyperglycemic spike.

Further, by predicting the timing at which the postprandial hyperglycemic spike occurs by referring to the past monitoring result representing the variation in the past blood glucose equivalent value of the user, it is possible to predict the timing at which the postprandial hyperglycemic spike occurs in consideration of the variation in the past blood glucose equivalent value of the user.

Further, by referring to the past meal time recorded on the time axis of the past monitoring result, it is possible to compare the past meal time with the latest meal time. Thereby, it is possible to predict the timing at which the postprandial hyperglycemic spike occurs in consideration of the variation in the past postprandial blood glucose equivalent value of the user.

In addition, by predicting the timing at which the postprandial hyperglycemic spike occurs based on the latest meal content of the user, it is possible to provide a notification of the blood sampling timing in consideration of the meal content.

In the embodiment, the user performs self blood sampling and measures the blood glucose value using a blood glucose measurement device. On the other hand, the present disclosure is not limited thereto. After the user performs self blood sampling, blood obtained by the blood sampling may be delivered to the test center, and a test may be performed at the test center. Alternatively, a test may be performed using a test device provided in a pharmacy, a station, a dedicated place (for example, a convenience store), or the like.

Further, in a case where a blood glucose equivalent value, an abnormal tendency, a selected test candidate, an image of a meal, and the like are stored in the test server 6, the stored information may be used as big data. The stored big data may be used for learning AI that provides information related to blood glucose values, or may be used as statistic information.

Further, in the embodiment, for example, in a case where a measured value of the blood glucose equivalent value is insufficient, an increase tendency of the blood glucose equivalent value may not be determined. For example, in a case where the user wears the measurement device 3 for a short time, the blood glucose equivalent value may not be monitored enough to determine the postprandial hyperglycemic spike. In such a case, the prediction unit 42 cannot determine an increase tendency of the blood glucose equivalent value. For this reason, in a case where the prediction unit 42 cannot determine an increase tendency of the blood glucose equivalent value, preferably, a notification urging the user to wear the measurement device 3 for a longer time is displayed. FIG. 16 is a diagram illustrating a notification screen. As illustrated in FIG. 16, on a notification screen 82, a notification 83 indicating “Please wear the measurement device for a longer time.” is displayed. From the notification screen 82, the user can take an action to wear the measurement device 3 for a longer time. Thereby, it is possible to determine an abnormal tendency of the blood glucose equivalent value after the action.

Further, in the embodiment, in a case where it is determined that the increase in the blood glucose equivalent value is not due to the postprandial hyperglycemic spike, any notification is not displayed. On the other hand, a notification may be displayed. FIG. 17 is a diagram illustrating a notification screen in a case where it is determined that the increase in the blood glucose equivalent value is not due to the postprandial hyperglycemic spike. As illustrated in FIG. 17, on a notification screen 84, a notification 85 indicating “A postprandial hyperglycemic spike does not occur. However, the blood glucose value tends to increase, so be aware of your lifestyle.” is displayed. Thereby, the user can recognize that there is no need to perform blood sampling.

Further, in the embodiment, the blood glucose equivalent value measured by the measurement devices 3 and 4 is transmitted to the mobile terminal 2, and the mobile terminal 2 notifies the user of the blood sampling timing. On the other hand, the present disclosure is not limited thereto. In particular, the test support program according to the present embodiment may be installed in the wristwatch-type measurement device 3, and the measurement device 3 may predict the timing at which a postprandial hyperglycemic spike occurs and provide a notification of the blood sampling timing. In this case, the measurement device 3 has a functional configuration of the test support apparatus according to the present embodiment illustrated in FIG. 5.

Further, in the embodiment, the notification unit 43 provides a notification of the blood sampling timing by displaying various notification screens on the touch panel 14. On the other hand, the present disclosure is not limited thereto. The notification unit 43 may present a test candidate to the user by voice.

Further, in the embodiment, the blood glucose equivalent value is measured by the measurement devices 3 and 4. On the other hand, the present disclosure is not limited thereto. Instead of measuring the blood glucose equivalent value by the measurement devices 3 and 4, a urine glucose value may be measured by performing a urine glucose test. In this case, the measured urine glucose value corresponds to the blood glucose equivalent value. In a case of the urine glucose test, since a urine glucose value is obtained by using a urine glucose test device, the urine glucose value may be used as a blood glucose equivalent value. The urine glucose test device is an example of a measurement device. In this case, the urine glucose test device has a communication function with the mobile terminal 2, and the measured urine glucose value is transmitted to the mobile terminal 2. Thereby, the mobile terminal 2 may monitor the blood glucose equivalent value based on the urine glucose value.

On the other hand, in a case where a urine glucose test is performed by using a test paper, a color of the test paper is changed according to a urine glucose value. In this case, an image of the test paper may be acquired by capturing the test paper by using the camera 18 of the mobile terminal 2, and the prediction unit 42 may recognize the color of the test paper from the image of the test paper. Thereby, an abnormal tendency may be determined. In this case, a measurement device is not required, and thus test support can be performed only by the mobile terminal 2. Therefore, the user does not need to prepare the measurement devices 3 and 4 or a urine glucose test device.

Further, in the embodiment, whether a meal recommended for blood sampling is breakfast, lunch, or dinner may be presented to the user by referring to the past monitoring result of the user. For example, in a case of the user having the past monitoring result 46 illustrated in FIG. 9, since the user does not have breakfast, preferably, as a meal recommended for blood sampling, lunch or dinner is presented to the user. In addition, in a case where it is determined that a peak of a postprandial hyperglycemic spike after breakfast is higher than peaks of postprandial hyperglycemic spikes after lunch and dinner by referring to the past monitoring result of the user, preferably, as a meal recommended for blood sampling, breakfast is presented to the user. Thereby, it is possible to predict a timing at which the peak of the postprandial hyperglycemic spike is likely to appear, and thus the user can prepare blood sampling. In addition, the user can recognize the timing at which his/her postprandial hyperglycemic spike is likely to appear.

Further, the blood glucose equivalent value may be monitored by using an ambulatory glucose profile (AGP) instead of the blood glucose equivalent value measured by the measurement devices 3 and 4. AGP is a useful analysis method for reading a tendency of a variation in the blood glucose value, that is, a blood glucose trend, from blood glucose values for several days obtained by continuous measurement or a glucose value in the interstitial fluid (https://dm-net.co.jp/trend/agp/001.php). By using AGP, it becomes easy to recognize a time zone in which hypoglycemia and hyperglycemia are likely to occur during a day and a time zone in which a variation in the blood glucose value is large. Therefore, by monitoring the blood glucose equivalent value by using AGP, it is possible to easily determine an abnormal tendency of the blood glucose equivalent value.

Further, in the embodiment, the blood glucose equivalent value measured by the measurement devices 3 and 4 is transmitted to the mobile terminal 2, and the mobile terminal 2 determines an abnormal tendency of the blood glucose equivalent value, determines an action candidate, and presents an action candidate. On the other hand, the present disclosure is not limited thereto. The blood glucose equivalent value measured by the measurement devices 3 and 4 may be transmitted to the test server 6 directly from the measurement devices 3 and 4 or via the mobile terminal 2, and the test server 6 may predict the timing at which a postprandial hyperglycemic spike occurs and provide a notification of the blood sampling timing. In this case, the test server 6 transmits the blood sampling timing to the mobile terminal 2 or the measurement device 3, and thus the mobile terminal 2 or the measurement device 3 can notify the user of the blood sampling timing.

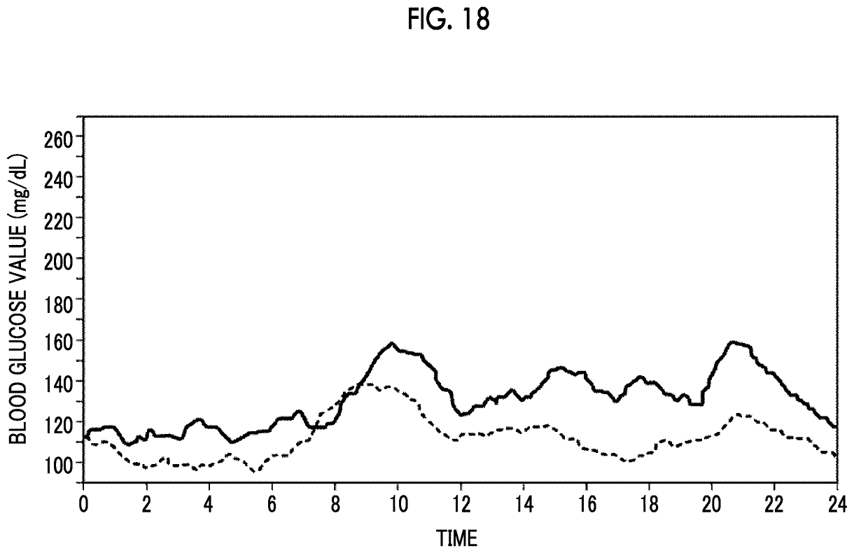

Further, in the embodiment, the blood glucose equivalent value measured by the measurement devices 3 and 4 is monitored, and thus a comparison result between a monitoring result of the blood glucose equivalent value and a test result obtained by blood sampling of the user may be presented to the user. FIG. 18 is a graph illustrating a comparison result between a monitoring result of the blood glucose equivalent value and a test result obtained by blood sampling of a user. In FIG. 18, a solid line represents a monitoring result of the blood glucose equivalent value, and a broken line represents a test result obtained by blood sampling. As illustrated in FIG. 18, the blood glucose equivalent value measured by the measurement devices 3 and 4 may be higher than the actual blood glucose value. As illustrated in FIG. 18, a comparison result between a monitoring result of the blood glucose equivalent value and a test result obtained by blood sampling of the user is presented to the user. Thereby, the user can determine a deviation between the current blood glucose equivalent value and the actual blood glucose value.

Further, in the embodiment, for example, as a hardware structure of processing units that execute various processing, such as the information acquisition unit 41, the prediction unit 42, and the notification unit 43, the following various processors are may be used. The various processors include, as described above, a CPU, which is a general-purpose processor that functions as various processing units by executing software (program), and a dedicated electric circuit, which is a processor having a circuit configuration specifically designed to execute a specific processing, such as a programmable logic device (PLD) or an application specific integrated circuit (ASIC) that is a processor of which the circuit configuration may be changed after manufacturing such as a field programmable gate array (FPGA).

One processing unit may be configured by one of these various processors, or may be configured by a combination of two or more processors having the same type or different types (for example, a combination of plural FPGAs or a combination of a CPU and an FPGA). Further, the plurality of processing units may be configured by one processor.

As an example in which the plurality of processing units are configured by one processor, firstly, as represented by a computer such as a client and a server, a form in which one processor is configured by a combination of one or more CPUs and software and the processor functions as the plurality of processing units may be adopted. Secondly, as represented by a system on chip (SoC) or the like, a form in which a processor that realizes the function of the entire system including the plurality of processing units by one integrated circuit (IC) chip is used may be adopted. As described above, the various processing units are configured by one or more various processors as a hardware structure.

Further, as the hardware structure of the various processors, more specifically, an electric circuit (circuitry) in which circuit elements such as semiconductor elements are combined may be used.

What is claimed is:

1. A test support apparatus comprising at least one processor, wherein the processor is configured to
predict a timing at which a postprandial hyperglycemic spike occurs by monitoring a variation in a blood glucose equivalent value of a user that correlates with a blood glucose value, and
provide a notification of at least one blood sampling timing for measuring the blood glucose value based on a result of the prediction.

2. The test support apparatus according to claim 1, wherein the processor is configured to
provide a notification of the blood sampling timing by using, as a reference, the timing at which the postprandial hyperglycemic spike occurs.

3. The test support apparatus according to claim 1, wherein the timing at which the postprandial hyperglycemic spike occurs is a timing at which the postprandial hyperglycemic spike reaches a peak.

4. The test support apparatus according to claim 1, wherein the processor is configured to
predict the timing at which the postprandial hyperglycemic spike occurs by referring to a past monitoring result representing a variation in a past blood glucose equivalent value of the user.

5. The test support apparatus according to claim 4, wherein the processor is configured to
predict the timing at which the postprandial hyperglycemic spike occurs by referring to a past meal time recorded on a time axis of the past monitoring result.

6. The test support apparatus according to claim 5, wherein the processor is configured to
predict the timing at which the postprandial hyperglycemic spike occurs by comparing, in the past monitoring result, the variation in the blood glucose equivalent value from the past meal time with the variation in the blood glucose equivalent value from a latest meal time of the user.

7. The test support apparatus according to claim 6, wherein the processor is configured to
provide, in a case where it is predicted that the postprandial hyperglycemic spike does not occur within a preset time from the latest meal time, a notification that the postprandial hyperglycemic spike does not occur.

8. The test support apparatus according to claim 5, wherein the processor is configured to
predict the timing at which the postprandial hyperglycemic spike occurs based on a latest meal content of the user.

9. The test support apparatus according to claim 8, wherein the processor is configured to
predict the timing at which the postprandial hyperglycemic spike occurs by referring to the latest meal content of the user and a past meal content of the user.

10. The test support apparatus according to claim 8, wherein the processor is configured to
determine the latest meal content by using an image of a meal of the user.

11. The test support apparatus according to claim 9, wherein the processor is configured to
determine the past meal content by using an image of a meal of the user.

12. The test support apparatus according to claim 10, wherein the image of the meal is a moving image.

13. The test support apparatus according to claim 1, wherein the processor is configured to
provide a notification of a plurality of the blood sampling timings for one timing at which the postprandial hyperglycemic spike occurs.

14. The test support apparatus according to claim 1, wherein the blood sampling timing is a timing at which the user has to perform self blood sampling by himself/herself.

15. The test support apparatus according to claim 1, wherein the blood glucose equivalent value is acquired by a measurement device attached to the user.

16. The test support apparatus according to claim 15, wherein the measurement device is a wearable device.

17. The test support apparatus according to claim 1, further comprising a measurement device that acquires the blood glucose equivalent value.

18. The test support apparatus according to claim 17, wherein the test support apparatus is a wearable apparatus.

19. A test support method comprising:
predicting a timing at which a postprandial hyperglycemic spike occurs by monitoring a variation in a blood glucose equivalent value of a user that correlates with a blood glucose value; and
providing a notification of at least one blood sampling timing for measuring the blood glucose value based on a result of the prediction.

20. A non-transitory computer-readable storage medium storing a test support program causing a computer to execute:
a procedure of predicting a timing at which a postprandial hyperglycemic spike occurs by monitoring a variation in a blood glucose equivalent value of a user that correlates with a blood glucose value; and
a procedure of providing a notification of at least one blood sampling timing for measuring the blood glucose value based on a result of the prediction.
